(19) United States
(12) Patent Application Publication
(10) Pub. No.: US 2023/0123717 A1
(43) Pub. Date: Apr. 20, 2023

(54) LOCALIZATION TARGET FOR A DIGITAL SURGICAL STEREOSCOPE

(71) Applicant: Digital Surgery Systems, Inc., Goleta, CA (US)

(72) Inventors: Thomas Kanusky, Goleta, CA (US); Stephen Minne, Goleta, CA (US); Alan Fridman, Goleta, CA (US); Norman Hannotte, Goleta, CA (US)

(21) Appl. No.: 17/967,406

(22) Filed: Oct. 17, 2022

Related U.S. Application Data
(60) Provisional application No. 63/256,466, filed on Oct. 15, 2021.

(51) Int. Cl. A61B 90/00; H04N 5/225

(52) U.S. Cl. CPC A61B 90/361; H04N 5/2251; A61B 2090/363; A61B 2090/367

(57) ABSTRACT

A localization target for a digital surgical stereoscope is disclosed herein. In an example, the localization target includes a shell apparatus for a surgical imaging camera. The apparatus includes a top surface integrally formed with a front surface and two opposing side surfaces defining empty space therebetween. Each of the top surface, the side surfaces, and the front surface includes at least three tracking features. The apparatus also includes at least six kinematic constraints located on an internally facing side of at least one of the top surface, the side surfaces, or the front surface. The apparatus further includes a connector that is positioned within an area defined by the at least six kinematic constraints.

### PRIORITY CLAIM

This application claims priority to and the benefit as a non-provisional application of U.S. Provisional Patent Application No. 63/256,466, filed Oct. 15, 2021, the entire contents of which are hereby incorporated by reference and relied upon.

### BACKGROUND

A surgical camera system typically includes an imaging camera (e.g., a stereoscopic camera) mounted on a robotic arm in addition to a computer system that controls the camera and positioning of the robotic arm. Oftentimes, the computer system includes a surgical navigation system that attempts to correlate or register a position of a live patient to a set of patient data (such as volumetric CT or MRI data). The navigation system also includes a means for detecting and displaying a position and orientation of the camera around and often inside the patient. The main goal of such a system is to provide a surgeon an approximate knowledge of where in the patient anatomy (which comprises a three-dimensional space) the surgeon is currently “looking” via the camera or a probe. A location of the probe tip is the primary object of interest when a traditional probe is used. In the case of a camera or microscope, the location of camera or microscope's focal point becomes the primary object of interest.

Known surgical navigation systems use a separate localizer or tracking camera to determine a transformation or orientation/position between the camera system and the patient. The localizer or tracking camera records the patient and the imaging camera in a same frame of reference to determine a relative position/orientation of each. The navigation system then registers the patient position/orientation to the imaging camera's position/orientation.

In an example, FIG. 1 is a diagram of a surgical navigation system 1 that includes an imaging camera 2 and a localizer 4. In this example, the localizer 4 is mounted separately from a robotic arm 6. In other instances, both the imaging camera 2 and the localizer 4 are connected to the robotic arm 6. Images recorded by the camera 2 are displayed via a monitor 8 for a surgeon.

To determine a position/orientation, the surgical imaging camera 2 and a patient are provided with a plurality of targets, fiducials, or cross points. A clamp 10 may be attached to a patient such that the targets, fiducials, or cross points are coupled to the clamp. Known positions of the targets, fiducials, or cross points relative to the clamp 10 enable images or data recorded by the localizer 4 to be transformed into a patient position/orientation. Similarly, a clamp 12 with targets, fiducials, or cross points may be attached to the imaging camera 2. As shown in FIG. 1, both of the clamps 10 and 12 have to be in a field of view of the localizer 4 to enable the registration between the patient and the imaging camera 2 to be determined throughout the surgical procedure. This configuration enables a surgical navigation system to register transformations between positions/orientations of the camera 2 and the patient even when a position or orientation of the camera 2 is changed using the robotic arm 6.

The use of the clamp 12 with the camera 2 can be problematic. Oftentimes, the targets, fiducials, or cross points of the camera clamp 12 are viewable from only a limited number of positions, which requires an optimal system setup prior to an operation, or requires position adjustments of the localizer 4 during the operation. Further, due to the adjustability of the clamps, the system generally requires calibration to determine a transformation or relationship between the targets, fiducials, or cross points of the image camera clamp 12 and an optical axis and/or focal point of the imaging camera 2.

Another known issue is that during an operation, sterility is critical. As such, a surgical drape is placed over the imaging camera 2 and the robotic arm 6. However, to enable the targets, fiducials, or cross points of the camera clamp 12 to be visible, holes or openings are cut into the drape. The presence of these holes is not ideal in a surgical environment. Further, to reduce the contamination from having the holes, the targets, fiducials, or cross points are often disposable and replaced after each operation. As one can imagine, frequently replacing targets, fiducials, or cross points is time consuming and costly.

Another known issue with the camera clamp 12 is the intrusiveness of the clamp itself. To maximize viewing angles for the localizer 4, the camera clamp 12 is typically mounted to project away from the imaging camera 2, as shown in FIG. 1. However, this projection away from the imaging camera 2 consumes vital surgical space. Moreover, its projection from the imaging camera 2 is prone to bumping from surgical staff, who are frequently handling surgical tools for a surgeon who is in close proximity to the imaging camera 2. One inadvertent bump of the camera clamp 12 may force a surgical team to have to recalibrate the entire surgical navigation system 1 during an operation, which is time consuming and potentially dangerous to the patient.

### SUMMARY

A localization target for a digital surgical stereoscope is disclosed herein. The localization target may include a shell apparatus that provides tracking for an imaging camera. The example shell apparatus is configured to be placed over a housing of an imaging camera, thereby sandwiching a surgical drape. The shell apparatus includes a housing configured as a target for a localizer or tracking camera. In some embodiments, the housing of the shell apparatus includes a top surface, side surfaces, and a front surface. Each of the surfaces includes at least three tracking features (e.g., targets, fiducials, or cross points) to enable a navigation system to determine a position/orientation of the imaging camera.

The housing of the shell apparatus also includes at least six kinematic constraints (e.g., three pairs of two constraints) for proper alignment with a housing of the imaging camera. Each pair of kinematic constraints, in some examples, is configured to engage at least one spherical or semi-spherical object on a top surface of the imaging camera. The positioning of the kinematic constraints ensures that the shell apparatus is properly positioned with respect to the imaging camera to within a few microns of error. This extremely small positioning error enables the shell apparatus to be registered to the imaging camera using known positions/orientations, thereby eliminating the need for calibration. Further, a relatively low profile of the shell apparatus provides more space for surgical staff and eliminates possibilities of being inadvertently bumped.

In light of the disclosure set forth herein, and without limiting the disclosure in any way, in a first aspect of the present disclosure, which may be combined with any other aspect, or portion thereof, described herein a shell apparatus for a surgical imaging camera includes a top surface integrally formed with a front surface and two opposing side surfaces defining empty space therebetween. Each of the top surface, the side surfaces, and the front surface includes at least three tracking features. The shell apparatus also includes at least six kinematic constraints located on an internally facing side of at least one of the top surface, the side surfaces, or the front surface. The shell apparatus further includes a connector that is positioned within an area defined by the at least six kinematic constraints.

In a second aspect of the present disclosure, which may be combined with any other aspect, or portion thereof, described herein, the at least six kinematic constraints are located on an underside of the top surface.

In a third aspect of the present disclosure, which may be combined with any other aspect, or portion thereof, described herein, the connector is located at the top surface.

In a fourth aspect of the present disclosure, which may be combined with any other aspect, or portion thereof, described herein, each of the top surface, the side surfaces, and the front surface is configured to cradle or at least partially cover a housing of the surgical imaging camera within the empty space.

In a fifth aspect of the present disclosure, which may be combined with any other aspect, or portion thereof, described herein, each of the at least six kinematic constraints is configured to mate or engage an object on a top surface of the surgical imaging camera.

In a sixth aspect of the present disclosure, which may be combined with any other aspect, or portion thereof, described herein, the object includes at least one of a spherical object or semi-spherical object.

In a seventh aspect of the present disclosure, which may be combined with any other aspect, or portion thereof, described herein, the connector is configured to engage a screw hole or receptor on the top surface of the surgical imaging camera.

In an eighth aspect of the present disclosure, which may be combined with any other aspect, or portion thereof, described herein, the top surface, the side surfaces, and the front surface are configured to sandwich a surgical drape between the surgical imaging camera and the shell apparatus.

In a ninth aspect of the present disclosure, which may be combined with any other aspect, or portion thereof, described herein, a shell apparatus for a surgical imaging camera includes a top surface integrally formed with a front surface and two opposing side surfaces defining empty space therebetween. Each of the top surface, the side surfaces, and the front surface includes at least three tracking features. The shell apparatus also includes at least six kinematic constraints located on at least one of the top surface, the side surfaces, or the front surface.

In a tenth aspect of the present disclosure, which may be combined with any other aspect, or portion thereof, described herein, the at least six kinematic constraints form points of a triangle.

In an eleventh aspect of the present disclosure, which may be combined with any other aspect, or portion thereof, described herein, each of the at least six kinematic constraints or grooves is configured to mate or engage a magnetic object on a respective surface of the surgical imaging camera to create a magnetic coupling between the surgical imaging camera and the shell apparatus.

In a twelfth aspect of the present disclosure, which may be combined with any other aspect, or portion thereof, described herein, the object includes at least one of a spherical object or semi-spherical object.

In a thirteenth aspect of the present disclosure, which may be combined with any other aspect, or portion thereof, described herein, the apparatus further includes at least one magnetic connector configured to magnetically couple to a corresponding at least one magnetic connector on the surgical imaging camera.

In a fourteenth aspect of the present disclosure, which may be combined with any other aspect, or portion thereof, described herein, the two opposing side surfaces each have a boot shape.

In a fifteenth aspect of the present disclosure, which may be combined with any other aspect, or portion thereof, described herein, at least one of the top surface, the side surfaces, or the front surface includes at least one window for weight reduction or heat dissipation.

In a sixteenth aspect of the present disclosure, which may be combined with any other aspect, or portion thereof, described herein, each of the at least six kinematic constraints are orientated at different angles with respect to each other.

In a seventeenth aspect of the present disclosure, which may be combined with any other aspect, or portion thereof, described herein, each of the top surface, the side surfaces, and the front surface includes protrusions configured to accept or support a respective tracking feature.

In an eighteenth aspect of the present disclosure, which may be combined with any other aspect, or portion thereof, described herein, each of the protrusions extend orthogonally from the respective surface.

In a nineteenth aspect of the present disclosure, which may be combined with any other aspect, or portion thereof, described herein, each of the protrusions and the respective tracking feature has a circular shape or an oval shape.

In a twentieth aspect of the present disclosure, which may be combined with any other aspect, or portion thereof, described herein, each of the tracking features includes at least one of a cross point, a fiducial, or a target.

In a twenty-first aspect of the present disclosure, which may be combined with any other aspect, or portion thereof, described herein, each of the top surface, the side surfaces, and the front surface are constructed from plastic, carbon fiber, fiberglass, nylon, metal, or composites thereof.

In a twenty-second aspect, any of the features, functionality and alternatives described in connection with any one or more of FIGS. 2 to 19 may be combined with any of the features, functionality and alternatives described in connection with any other of FIGS. 2 to 19.

In light of the present disclosure and the above aspects, it is therefore an advantage of the present disclosure to provide a shell apparatus that includes tracking features to enable a surgical navigation system to register a surgical imaging camera with a patient position/orientation.

It is another advantage of the present disclosure to provide a shell apparatus that enables a surgical imaging camera to be draped for operation room sterility while enabling surgical navigation tracking.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of a surgical navigation system that includes an imaging camera and a localizer.

FIG. 2 is a diagram of a shell apparatus, according to an example embedment of the present disclosure.

FIG. 3 is a diagram of a perspective view of the shell apparatus of FIG. 2, according to an example embodiment of the present disclosure.

FIG. 4 shows a front-view of the shell apparatus of FIG. 2, according to an example embodiment of the present disclosure.

FIG. 5 shows a side-view of the shell apparatus of FIG. 2, according to an example embodiment of the present disclosure.

FIG. 6 is a diagram of a top-plan view of the shell apparatus of FIG. 2, according to an example embodiment of the present disclosure.

FIG. 7 is a bottom-up view of the shell apparatus of FIG. 2, according to an example embodiment of the present disclosure.

FIG. 8 is a diagram of a bottom perspective of the shell apparatus of FIG. 2, according to an example embodiment of the present disclosure.

FIG. 9 is a diagram of a front perspective-view of the shell apparatus of FIG. 2, according to an example embodiment of the present disclosure.

FIG. 10 is a diagram of the imaging camera before placement of the shell apparatus, according to an example embodiment of the present disclosure.

FIG. 11 is an image of the imaging camera after placement of the shell apparatus, according to an example embodiment of the present disclosure.

FIG. 12 is a diagram that shows the coupling between a pair of kinematic constraints and an object, such as a ball bearing, according to an example embodiment of the present disclosure.

FIG. 13 is a diagram showing that when at least three kinematic constraint pairs are used, movement of a plate or the apparatus is restricted along multiple axes, including angular axes, according to an example embodiment of the present disclosure.

FIG. 14 is another diagram of a coupling triangle formed by the kinematic constraint pairs, according to an example embodiment of the present disclosure.

FIGS. 15 and 16 are diagrams of connectors used to couple the shell apparatus to an imaging camera, according to example embodiments of the present disclosure.

FIGS. 17 and 18 are diagrams of an alternative embodiment of the shell apparatus, according to an example embodiment of the present disclosure.

FIG. 19 is a diagram of another embodiment for camera tracking, according to an example embodiment of the present disclosure.

### DETAILED DESCRIPTION

A shell apparatus is disclosed herein that provides tracking for an imaging camera. The example shell apparatus is configured to be placed over a housing of an imaging camera, thereby sandwiching a surgical drape. The shell housing is a target for a localizer or tracking camera and has at least at least one surface. In some embodiments, the shell apparatus includes a top surface, side surfaces, and a front surface. Each of the surfaces includes at least three tracking features (e.g., targets, fiducials, or cross points) to enable a surgical navigation system to determine a position/orientation of the imaging camera.

The shell apparatus includes at least six kinematic constraints (e.g., three pairs of two constraints) for proper alignment with the housing of the imaging camera. Each pair of kinematic constraints, in some examples, is configured to engage at least one spherical or semi-spherical object on a top surface of the imaging camera. In other examples, the kinematic constraints may be singularly (not in pairs) placed to eliminate (or control) the 6 degrees of freedom along x, y, and z axes and rotations around those axes to deterministically place the shell apparatus on the imaging camera. In other examples, one group may include three kinematic constraints that form a trihedral pocket, two kinematic constraints that form a groove, and a flat kinematic constraint. It should be appreciated that virtually any combination of six or more (or five or less) kinematic constraints may be used for the shell apparatus.

Regardless of configuration, the positioning of the kinematic constraints ensures that the shell apparatus is properly positioned with respect to the housing of the imaging camera to within a few microns of error. This extremely small positioning error enables the shell apparatus to be registered to the imaging camera using known positions/orientations, thereby eliminating the need for calibration. Further, a relatively low profile of the shell apparatus provides more space for surgical staff and eliminates possibilities of being inadvertently bumped.

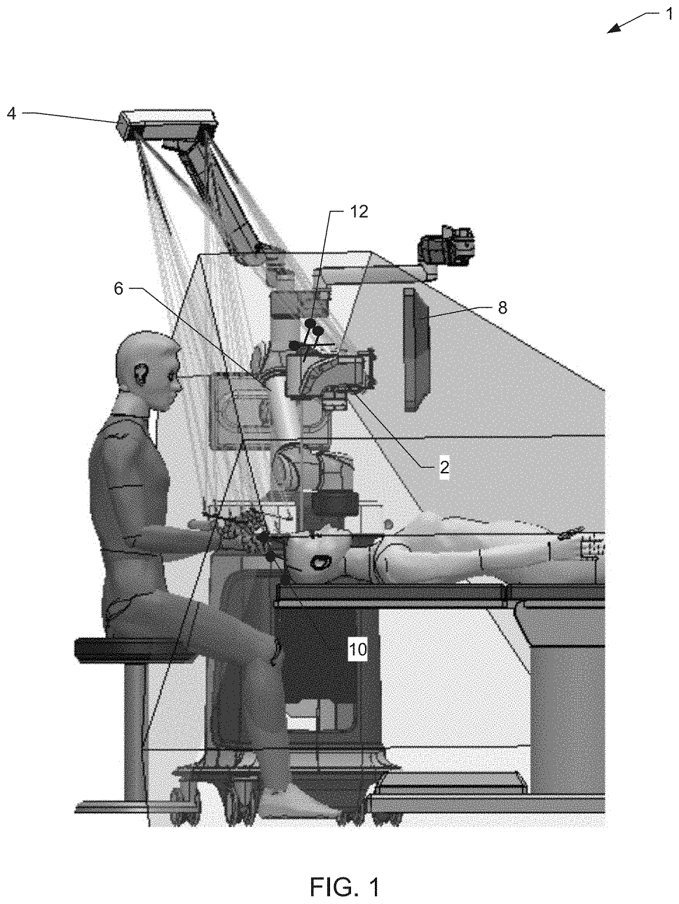

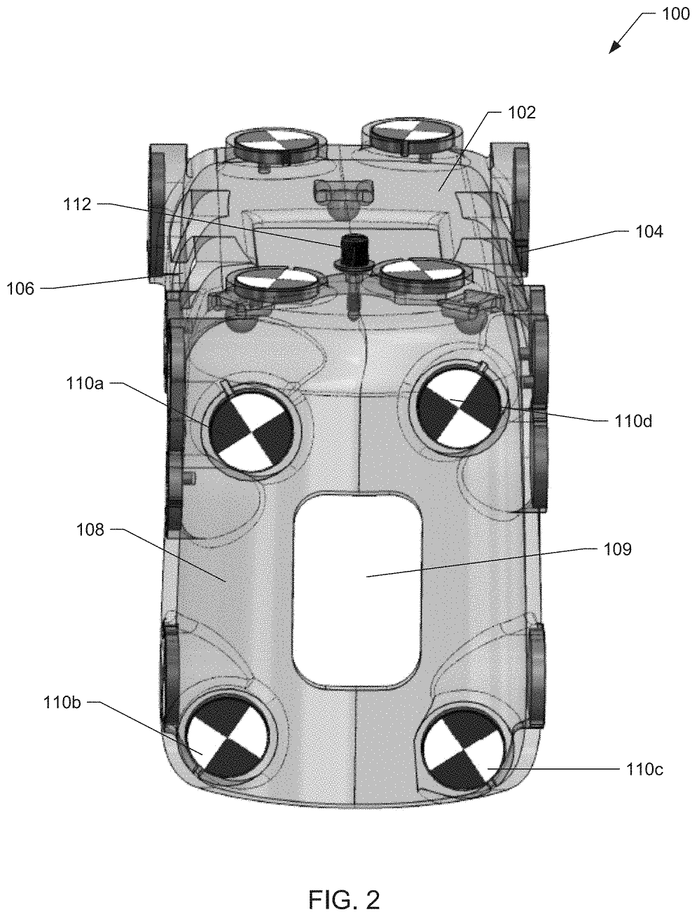

FIG. 2 is a diagram of a shell apparatus 100, according to an example embedment of the present disclosure. The shell apparatus 100 includes a top surface 102 and opposing side surfaces 104 and 106. The shell apparatus 100 also includes a front surface 108. Each of the surfaces 102 to 108 may be integrally formed or otherwise connected together. As disclosed herein, interiors of the surfaces 102 to 108 define an empty space, which is configured to receive an imaging camera, such as the imaging camera 2 of FIG. 1. The surfaces 102 to 108 accordingly partially cover or cradle at least a top surface, side surfaces, and a front surface of the imaging camera 2.

Each of the surfaces 102 to 108 includes at least one tracking feature 110. Preferably, to enable proper tracking using the localizer 4, each surface 102 to 108 includes at least three tracking features 110. For instance, FIG. 2 shows the front surface 108 with four tracking features 110a, 110b, 110c, and 110d. Each of the tracking features 110 are provided at some offset from each other to enable localization based on positioning and/or orientation.

In the illustrated example, each of the tracking features 110 includes a cross point, which may include an anodized block aluminum laser marked disc and/or oval. The tracking features 110 could also be integral to the shell apparatus 100. For example, the tracking features 110 could be painted on the shell apparatus 100. In an embodiment, the shell apparatus 100 may be anodized or ceramic coated, with laser engraving to create the tracking features 110.

As shown in FIG. 2, each disk or oval cross point is portioned into quarters, alternating between dark and light shading. Each of the disks or ovals has the crossing at slightly different angles to provide for identification when used in combination with spacing between each other. While cross points are shown throughout, it should be appreciated that the shell apparatus 100 may alternatively or additionally include fiducials or targets.

In some embodiments, the shell apparatus 100 includes a connector 112 that is positioned within a through hole of the top surface 102. The connector 112 may include a screw, a double stud, a single stud, and/or an adjustable torque thumb screw or knob. The connector 112 is configured to engage a corresponding screw hole in a top surface of the imaging camera 2 to connect or secure the shell apparatus 100 to a housing of the imaging camera 2.

In alternative embodiments, the connector 112 may be located on one or more of the side surfaces 104 and 106. It should be appreciated that the connector 112 provides a locking force between the shell apparatus 100 and the camera 2. The connector 112 is configured to provide a force that is aligned normal to the contact between the shell apparatus 100 and the camera 2. As such, the connector 112 is configured to be placed within an area defined by the kinematic constraints, as discussed below.

Further, while a male connector 112 is shown and described, in other embodiments, the connector 112 may include a female connector (e.g., a screw hole) that is configured to receive a screw or other male connector that is part of the top surface of the imaging camera 2.

In yet alternative embodiments, the connector 112 may be omitted. In these examples, the shell apparatus 100 may include magnetic couplers, such as kinematic constraints or grooves that mate with objects on the top surface of the imaging camera 2.

The shell apparatus 100 may be formed of polymers, carbon fiber, fiberglass, metal, or combinations thereof. The shell apparatus 100 (including the tracking features 110 and the connector 112) are configured to have a weight, when combined with the imaging camera 2, that is less than 5.5 kilograms. In some embodiments, the shell apparatus 100 itself may have a weight that is between 0.5 to 2.0 kilograms. The relatively low weight of the shell apparatus 100 prevents overloading of the robotic arm 6. To reduce weight, at least one of the surfaces 102 to 108 may include one or several windows similar to window 109 or other cutout. The window 109 may be any shape and/or size. The shell apparatus 100 is configured to be formed of a material that retains its rigidity even with the windows 109 in one or more of the surfaces 102 to 108.

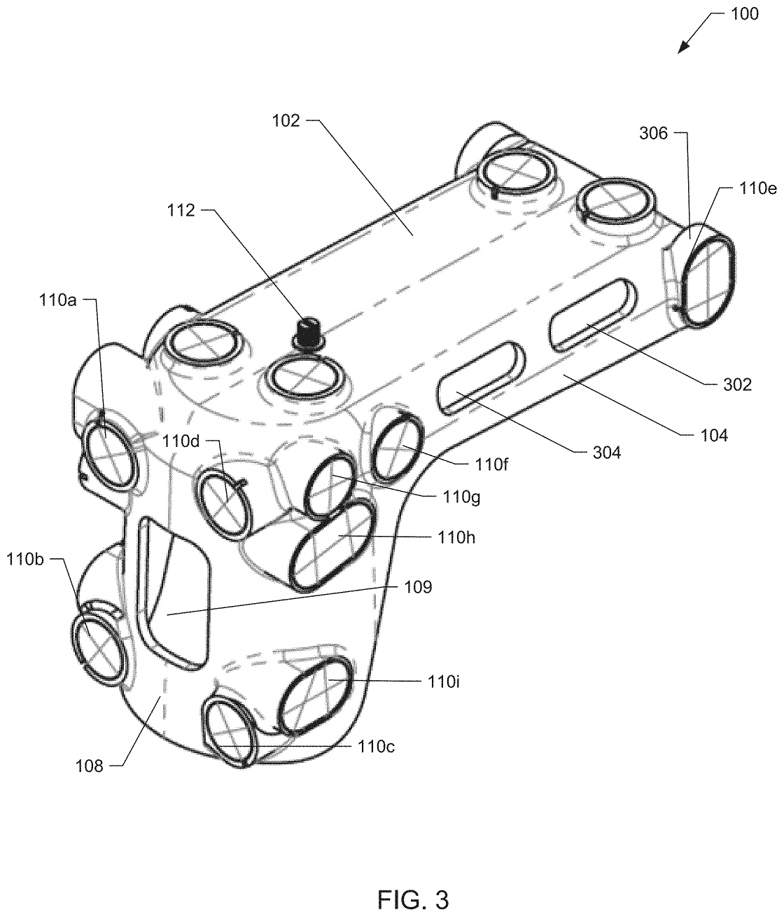

FIG. 3 is a diagram of a perspective view of the shell apparatus 100 of FIG. 2, according to an example embodiment of the present disclosure. In this example, the side surface 104 is more readily visible. As shown, the side surface 104 has a boot-shape that corresponds to a shape of the imaging camera 2. The side surface 104 includes tracking features 110e, 110f, 110g, 110h, and 110i. The tracking features 110f and 110g have a circular shape, while the tracking features 110e, 110h, and 110i have an oval shape. In other examples, the tracking features 110 may include other shapes, such as squares, triangles, rectangles, pentagons, hexagons, etc.

FIG. 3 shows that the side surface 104 includes two windows 302 and 304 to reduce weight and/or provide for heat dissipation. The windows 302 and 304 are provided between the tracking features 110e and 110f, but may be provided in other locations in alternative embodiments. In yet other embodiments, the windows 302 and 304 may be omitted or the side surface 104 may include a single window.

FIG. 3 also shows that the tracking features 110 are located within or on projections 306 of the respective surfaces 102 to 108. For example, a projection 306 is provided on an end of the side surface 104. The projection 306 extends orthogonally from the side surface 104. The projection 306 includes a recessed center section, which receives or otherwise accepts the tracking feature 110e. In some embodiments, a center of the projection 306 may not be recessed such that that tracking features 110 are provided at a surface of the projections. In yet alternative embodiments, the projections 306 may be omitted.

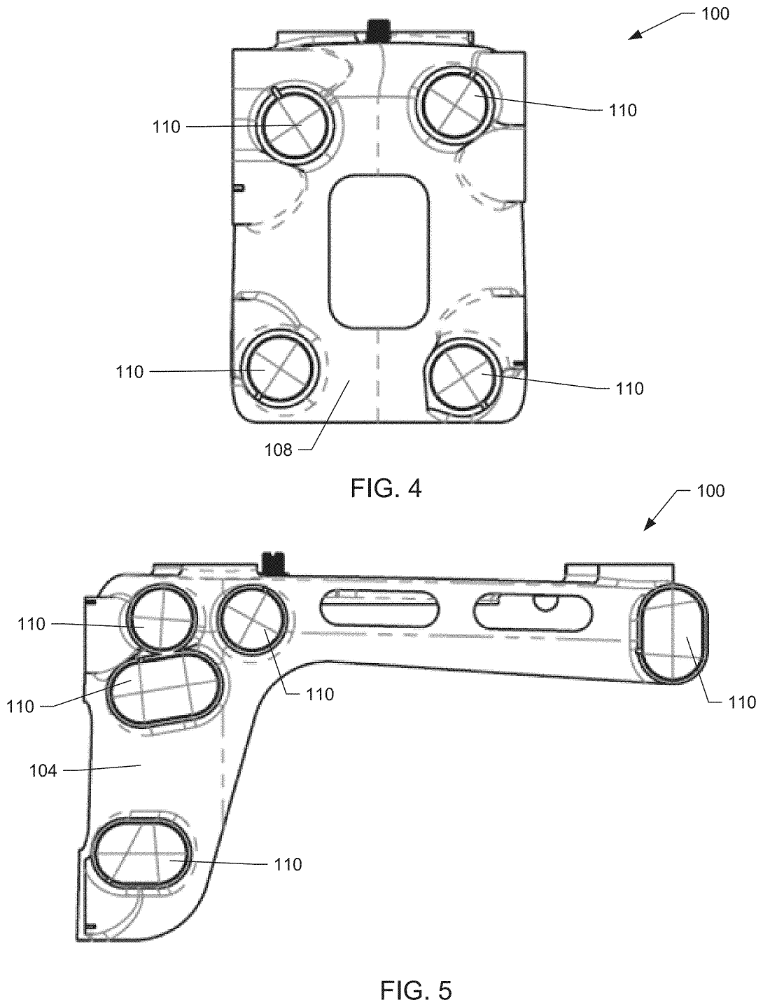

FIG. 4 shows a front-view of the shell apparatus 100, while FIG. 5 shows a side-view of the shell apparatus 100, according to an example embodiment of the present disclosure. As shown in FIG. 4, the front surface 108 has an approximately rectangular shape with four tracking features 110. FIG. 5 shows that the side surface 104 has a boot or L-shape and has five tracking features 110 to provide for tracking of the unique shape. The oval-shaped tracking features 110 each includes two cross points while the circular tracking features each include one cross point. This configuration provides eight separate items for tracking. The side surface 106 is a mirror image of the side surface 104 of FIG. 5.

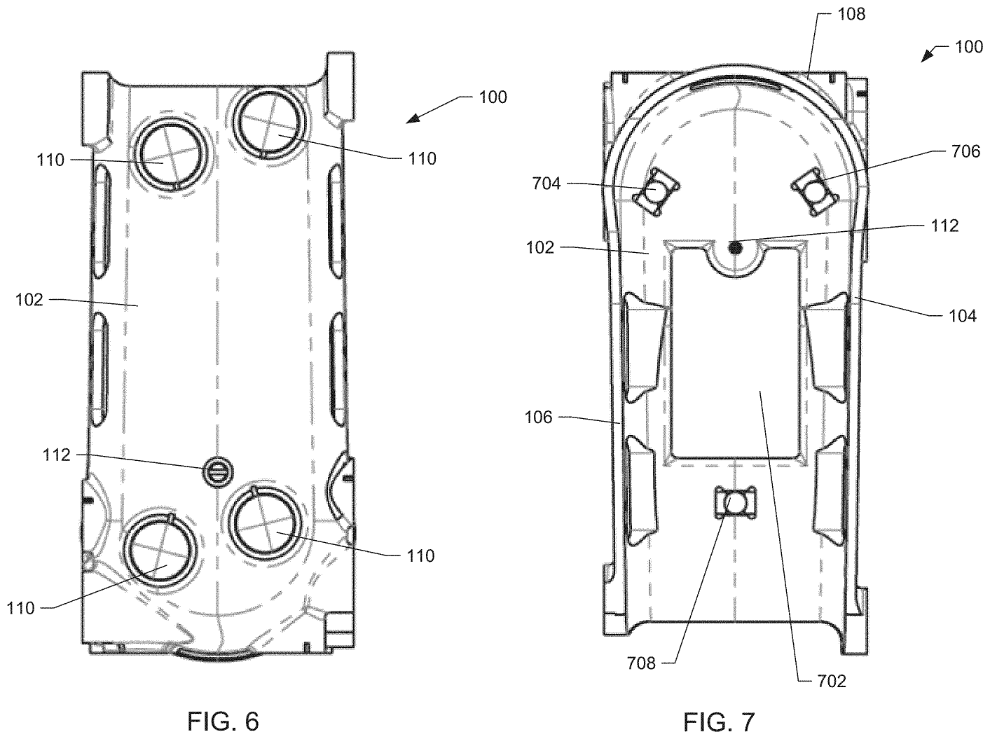

FIG. 6 is a diagram of a top-plan view of the shell apparatus 100, while FIG. 7 is a bottom-up view of the shell apparatus 100, according to an example embodiment of the present disclosure. FIG. 6 shows four tracking features 110 located on the top surface 102. FIG. 6 also shows a position of the connector 112 relative to the tracking features 110. FIG. 7 shows an underside of the top surface 102. Interiors of the surfaces 104, 106, and 108 are also visible. Interiors of the surfaces 102 to 108 for an opening that matches a shape of a housing of the imaging camera 2. The connector 112 is configured to couple the shell apparatus 100 to the camera 2.

FIG. 7 also shows that the interior of the top surface 102 may include a rib or plate 702 for structural support and/or heat dissipation. While a rectangular plate 702 is shown, in other examples, narrow ribs may be used. In other embodiments, the rectangular plate 702 is omitted.

The illustrated embodiment also shows three pairs of kinematic constraint features 704, 706, and 708, resulting in six total constraints that are located on an underside of the top surface 102. Each pair of constraints 704 to 708 includes two separate constraints configured to engage an object on a top surface of the imaging camera 2. The object may include a spherical or semi-spherical object, such as a ball bearing. The positioning of the kinematic constraints 704, 706, and 708 is configured to ensure the shell apparatus 100 is properly aligned with a housing of the imaging camera 2, with as few as a few microns of error in positioning. The precise positioning is important to ensure a known registration between the shell apparatus 100 and the imaging camera 2 can be used for surgical navigation without the need to calibrate before every surgical operation. While three pairs of kinematic constraints are shown, in other examples the top and/or side surfaces 102 to 108 may include additional kinematic constraints. For example, the top surface 102 and each of the side surfaces 104 and 106 may each include three kinematic constraints. Together, these nine constraints may rely on elastic averaging for precise alignment of the shell apparatus 100 with the camera 2.

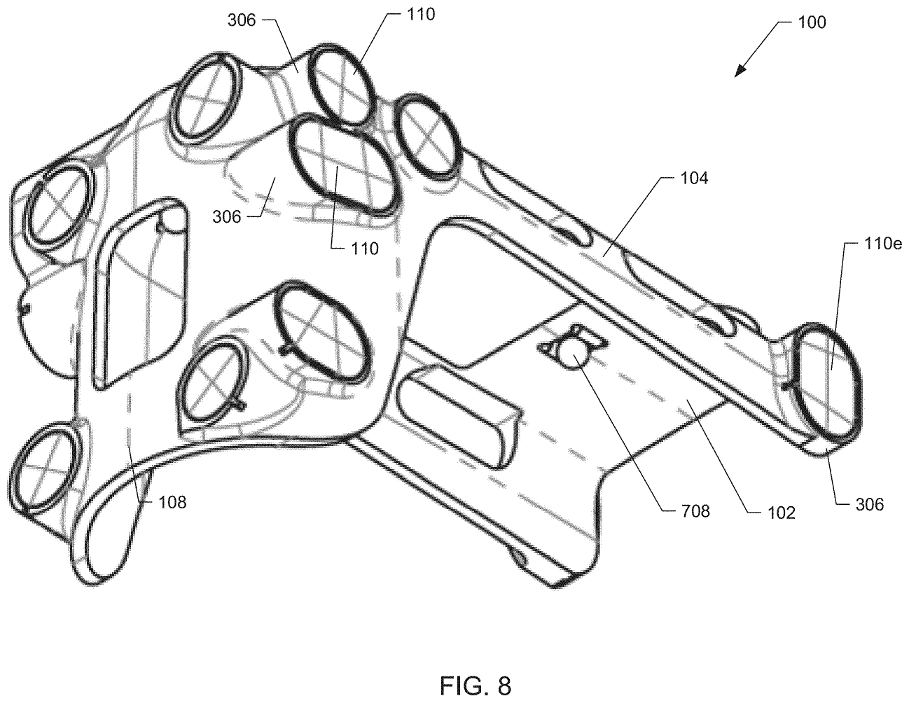

FIG. 8 is a diagram of a bottom perspective of the shell apparatus 100, according to an example embodiment of the present disclosure. The kinematic constraint 708 is visible in addition to an edge of the front surface 108. As shown, the front surface 108 has a curvature that conforms to a front surface of the camera 2. The curvature also provides for seamless integration of the front surface 108 with the side surfaces 104 and 106. Projections 306 of at least some of the tracking features 110 are also readily viewable.

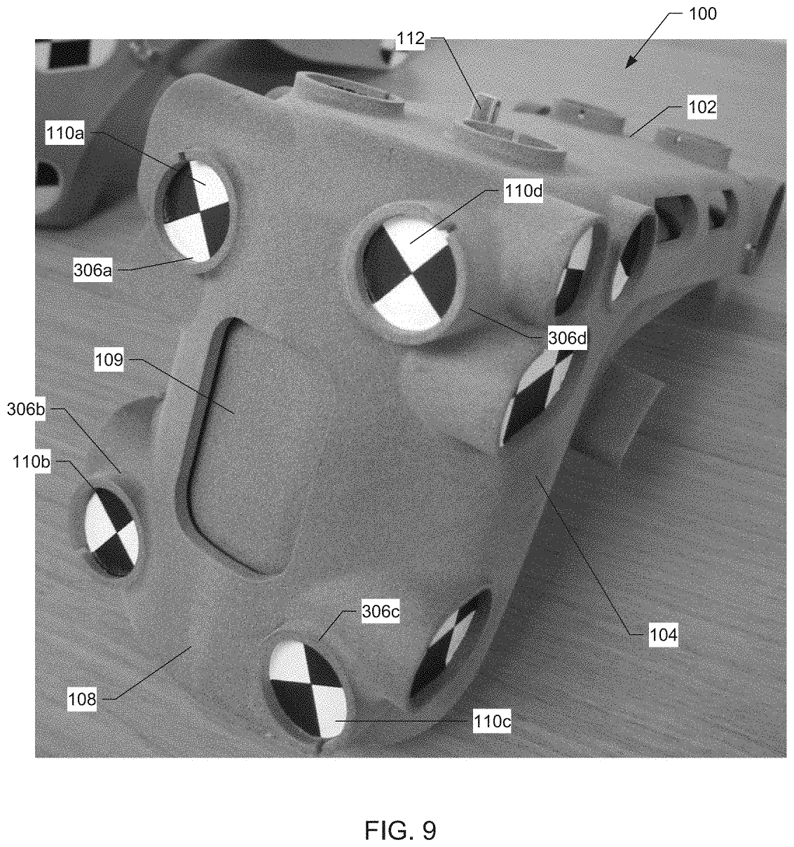

FIG. 9 is a diagram of a front perspective-view of the shell apparatus 100, according to an example embodiment of the present disclosure. The diagram shows the four tracking features 110a to 110d of the front surface 108. Further, the image shows projections 306a to 306d for each of the tracking features 110a to 110d. Center recess sections of the projections 306a to 306d (and other projections on the side surface 104 and the top surface 102) are also viewable.

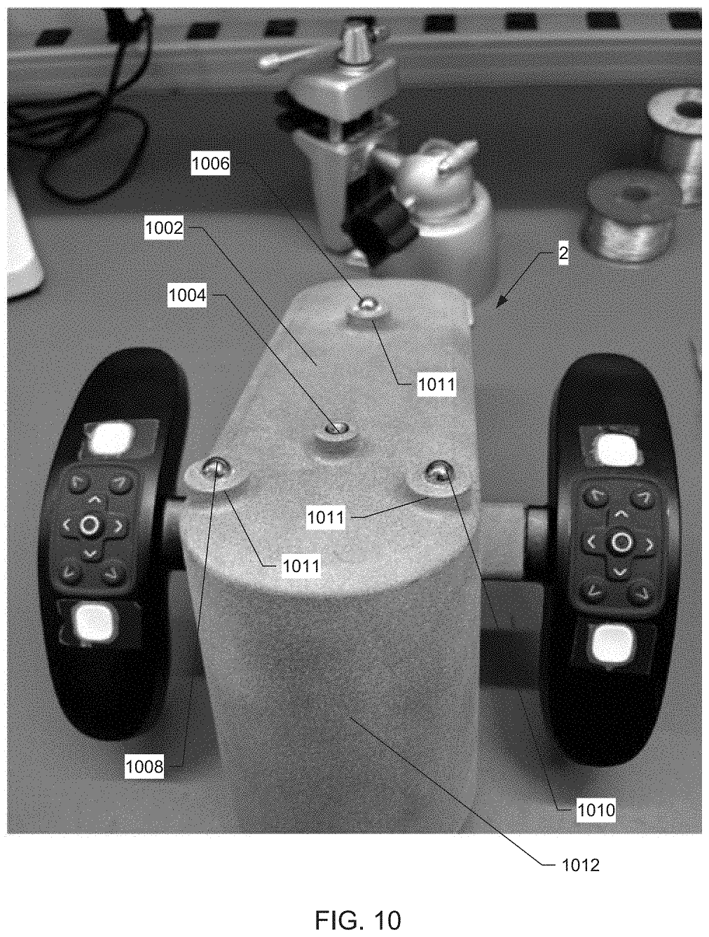

FIG. 10 is an image of the imaging camera 2 before placement of the shell apparatus 100, according to an example embodiment of the present disclosure. The imaging camera 2 includes a top surface 1002, which comprises a screw hole or other receptacle 1004 for receiving the connector 112 of the shell apparatus 100. In other embodiments, the screw hole or other receptacle 1004 may include a male connector. The top surface 1002 also includes spherical or semi-spherical objects 1006, 1008, and 1010. Each of the objects 1006 to 1010 is configured to mate with the corresponding kinematic constraints 704 to 708 of FIG. 7. As shown, the screw hole or other receptacle 1004 is provided in an area defined by the objects 1006 to 1010 such that a contact force is applied between the kinematic constraints. In some embodiments, the location of the screw hole or other receptacle 1004 (and corresponding connector 112) is selected to evenly distribute a contact force among the objects 1006 to 1010 and corresponding kinematic constraints 704 to 708.

In some embodiments, the objects 1006 to 1010 and/or the kinematic constraints 704 to 708 may be magnetic to enable magnetic coupling. In these embodiments, the connector 112 and the screw hole or other receptacle 1004 may be omitted.

FIG. 10 also shows that the screw hole or other receptacle 1004 and the objects 1006 to 1010 are supported by their own projections 1011 from the top surface 1002 of the imaging camera 2. The use of the projections 1011 helps ensure the objects 1006 to 1010 mate properly with the kinematic constraints 704 to 708 without surface features of the top surface 1002 of the housing of the camera 2 interfering.

FIG. 10 also shows that a front face 1012 of the housing of the camera 2 has a curvature. As shown in FIG. 8, the curvature of the front face 108 of the apparatus 100 corresponds to the curvature of the front face 1012 of the camera 2. Such a configuration enables the apparatus 100 to cradle the camera 2.

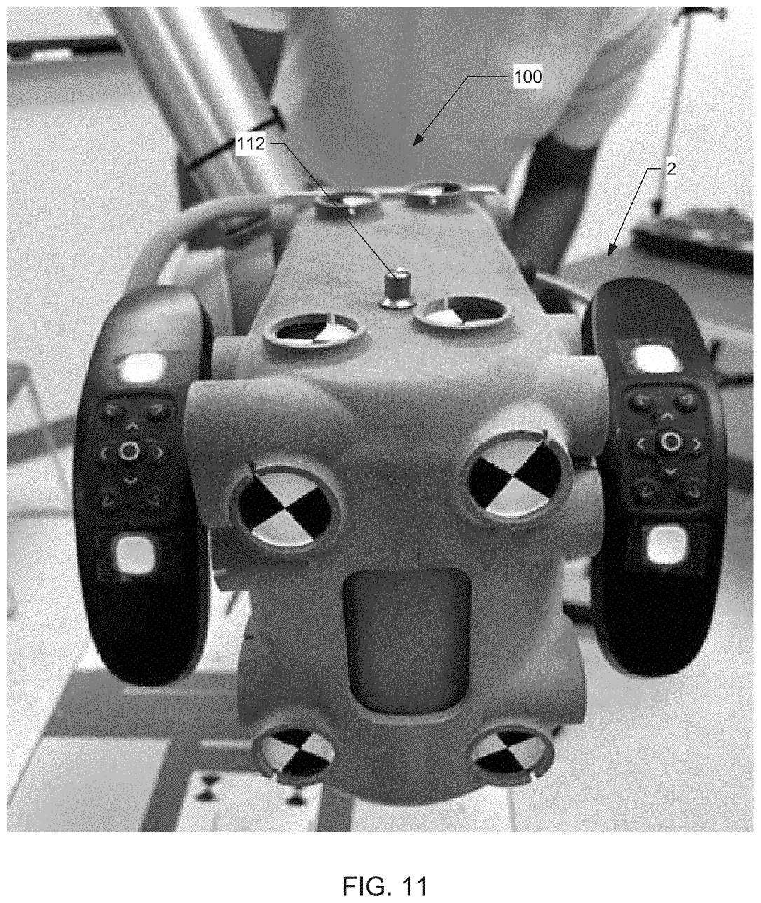

FIG. 11 is a diagram of the imaging camera 2 after placement of the shell apparatus 100, according to an example embodiment of the present disclosure. In this example, the kinematic constraints 704 to 708 have engaged the objects 1006 to 1010 for properly aligning the shell apparatus 100 with the camera 2. Further, the connector 112 is engaged with the screw hole or other receptacle 1004 of the camera 2. In operation, a surgical drape is sandwiched between the shell apparatus 100 and the camera 2. A small hole may be cut into the surgical drape to enable the connector 112 to mate with the screw hole or other receptacle 1004. However, separate holes are not needed between the kinematic constraints 704 to 708 and the respective the objects 1006 to 1010. The connection is secure enough such that any movement of the drape will not dislodge the apparatus 100.

As shown in FIG. 11, the shell apparatus 100 has a relatively low profile while providing four surfaces with tracking features for localizer visibility. This enables the camera 2 to be used in tight and difficult positions without consuming needed surgical space. This also ensures a superior line of sight with the localizer regardless of positioning. After each operation, the shell apparatus 100 may be removed from the camera 2 and cleaned/disinfected before the next use.

While FIGS. 2 to 11 show four surfaces 102 to 108 of the shell apparatus 100, it should be appreciated that fewer or additional surfaces may be used. For example, as few as one surface may be sufficient for some applications. Alternatively, two or three surfaces may be sufficient to cradle a camera.

Further, while the kinematic constraints 704 to 708 are described as being located on an underside of the top surface 102, in other embodiments they may be located on interior sides of one or more of the surfaces 102 to 108. For example, the kinematic constraint 704 may be located on an underside of the top surface 102, the kinematic constraint 706 may be located on an inside of the side surface 104, and the kinematic constraint 708 may be located on an inside of the side surface 106. In these embodiments, the objects 1006 to 1010 are mounted on corresponding surfaces of the camera 2. As one can appreciate, there are virtually endless configurations of kinematic constraints and surfaces to secure the shell apparatus 100 to the camera 2.

While FIGS. 2 to 11 show the kinematic constraint pairs 704, 706, and 708 as being located on the shell apparatus 100, in other embodiments the kinematic constraint pairs 704, 706, and 708 may be located on the surface 1002 of the camera 2. In these embodiments, the objects 1006 to 1010 may be provided on an underside of the top surface 102 of the apparatus 100.

### Kinematic Coupling Embodiment

As discussed above, the shell apparatus 100 includes kinematic constraints 704 to 708 that mate with objects 1006 to 1010 on the camera 2 to provide a precise alignment that is needed for surgical navigation registration. Even errors greater than 20 to 40 microns can significantly degrade registration and hinder surgical navigation of the camera 2 using a frame of reference of a patient.

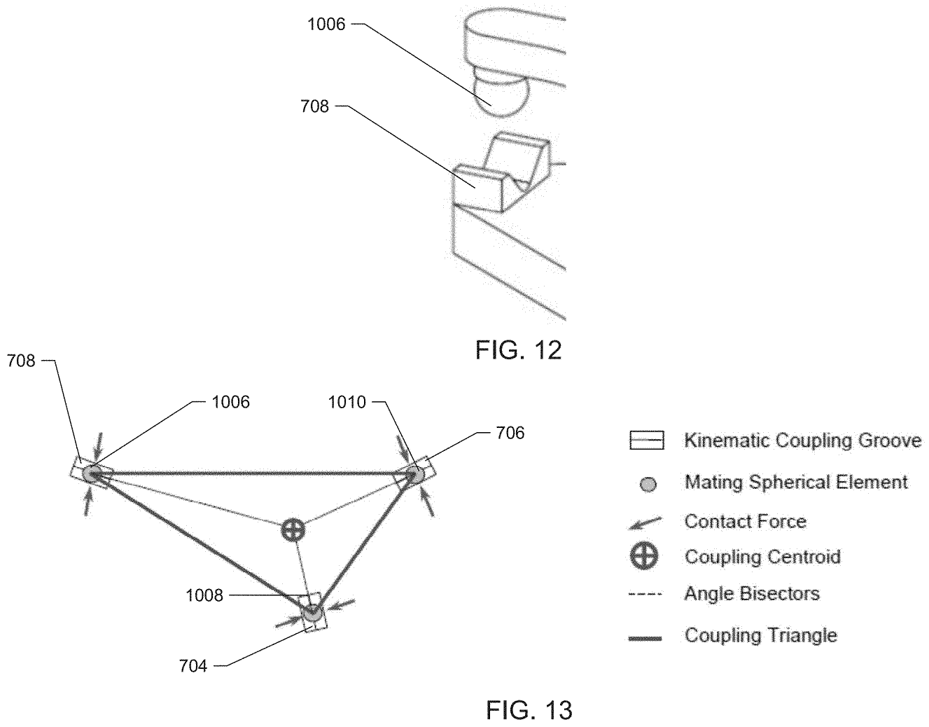

FIG. 12 is a diagram that shows the coupling between a pair of kinematic constraints 708 and an object 1006, such as a ball bearing, according to an example embodiment of the present disclosure. The object 1006 is configured to be placed between the kinematic constraints 708. Faces of the kinematic constraints 708 are slanted or angled, causing the object 1006 to be placed therebetween. The kinematic constraints 708 provide a contact force along one axis of movement, thereby preventing the object 1006 from moving along that axis. However, the kinematic constraints 708 do not restrict movement in other axes.

FIG. 13 shows that when at least three kinematic constraint pairs 704, 706, and 708 are used, movement of a plate or the apparatus 100 is restricted along multiple axes, including angular axes. Together, the kinematic constraint pairs 704, 706, and 708 form a coupling triangle with a coupling centroid. Such a configuration prevents motion in an orthogonal direction relative to the coupling triangle. Further, such a configuration provides near-perfect positioning down to a few microns.

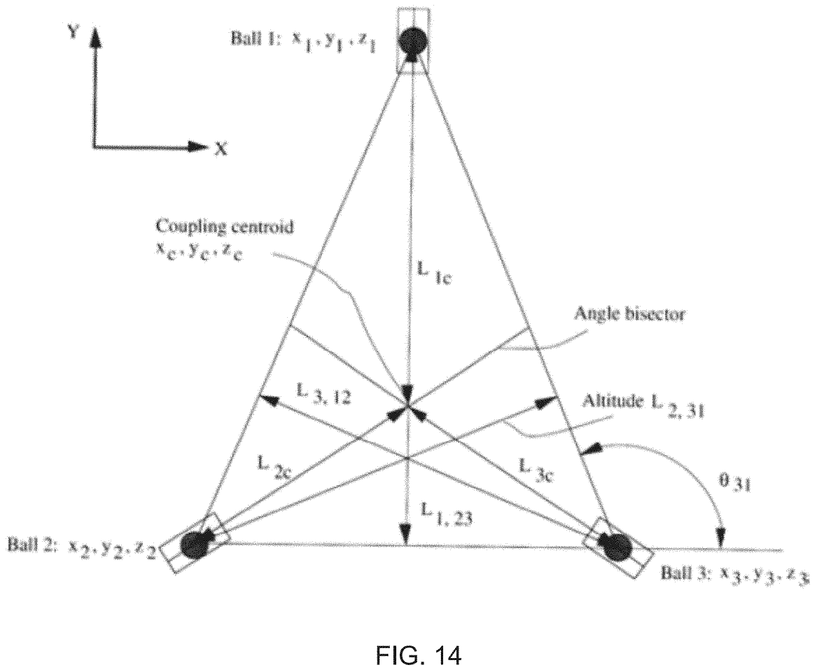

FIG. 14 is another diagram of a coupling triangle formed by the kinematic constraint pairs 704, 706, and 708. As shown, each kinematic constraint pair 704, 706, and 708 restricts movement along some axis. When used in combination, the kinematic constraint pairs 704, 706, and 708 restrict any in-plane movement or rotation. It should also be appreciated that a location of the coupling plane is important to avoid sine errors, and that for good stability, normal to planes containing contact for vectors should bisect angles of the coupling triangle. Further, a coupling triangle centroid lies at a center circle that coincides with centers of the three objects 1006 to 1010. The coupling centroid is also at an intersection of angle bisectors. The mounting of the objects 1006 to 1010 at different radii makes the design crash-proof. Further, non-symmetric groove placement makes the coupling user-proof.

### Connector Embodiment

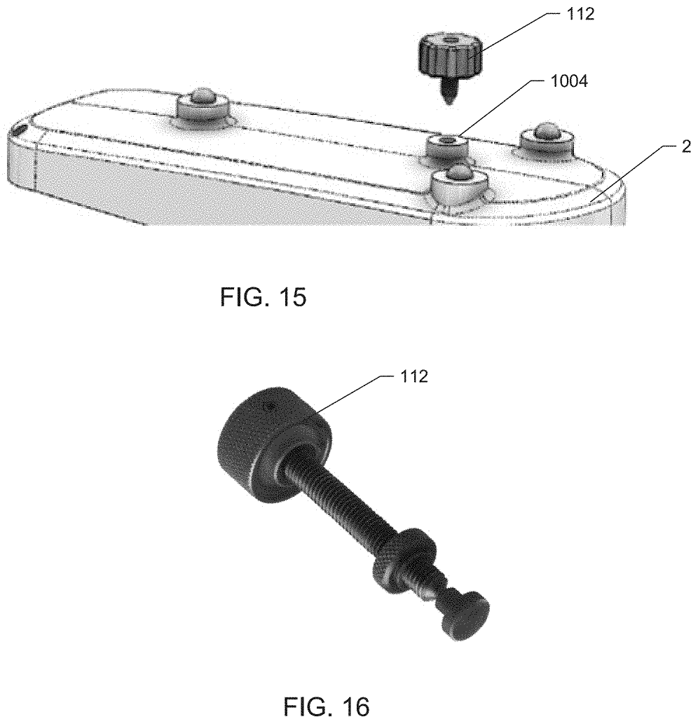

As discussed above, the connector 112 of the shell apparatus 100 includes a screw or other male connector. FIG. 15 is a diagram of the connector 112 shown as a single stud, according to an example embodiment of the present disclosure. The single stud 112 includes a knob that enables user actuation. The single stud 112 passes through the top surface 102 of the apparatus 100 to mate with the screw hole 1004 of the camera 2. In this illustrated example, the apparatus 100 is hidden for clarity.

FIG. 16 is a diagram of the connector 112 show as an adjustable torque thumb screw. The knurled-head thumb screw exerts positive, repeatable force between the apparatus 100 and the camera 2. When turned clockwise, the knob declutches and turns freely until a desired torque is reached. When turned counterclockwise, the knob locks for positive retraction. To set a desired force, first an outer cover screw at the side of the knob is removed, which exposes an adjustment screw. Next, a hex wrench is inserted to turn the adjustment screw until desired torque is achieved. The outer screw cover can then be re-installed. The torque can be adjusted from 3 to 6 inch-lbs, resulting in a contact force of 10 to 125 lbs.

### Alternative Shell Apparatus Embodiments

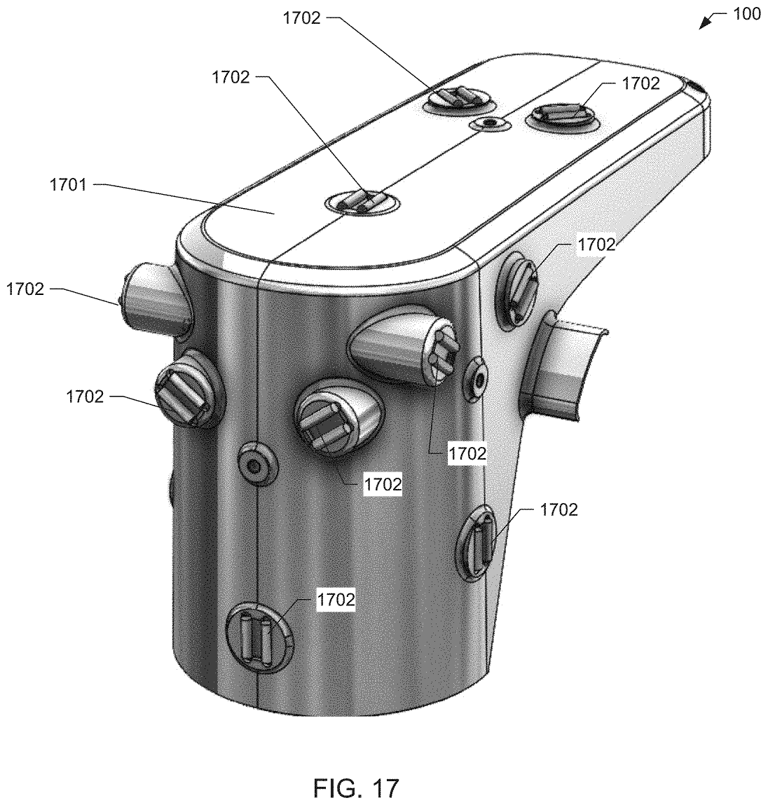

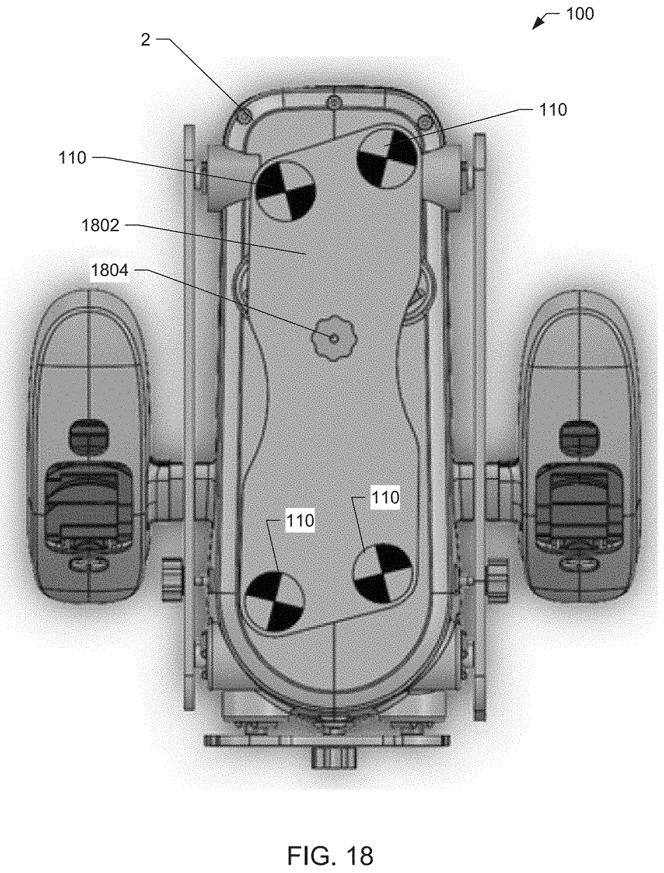

FIGS. 17 and 18 are diagrams of an alternative embodiment of the shell apparatus, according to an example embodiment of the present disclosure. In this embodiment, the shell apparatus is a cover or housing 1701 of the camera 2 that includes kinematic constraint pairs 1702 on surfaces. Each surface includes at least three kinematic constraint pairs.

As shown in FIG. 18, a plate 1802 includes spherical objects that mate or engage with each of the kinematic constraint pairs 1702. The plate 1802 also includes a connector 1804 for connection to the respective surface of the cover 1701. The plate 1802 also includes at least three tracking features 110. In this example, the plate 1802 is connected to the top surface 102 of the cover 1701. Separate plates may also be connected respectively to the side surfaces 104 and 106 and the front surface of the cover 1701.

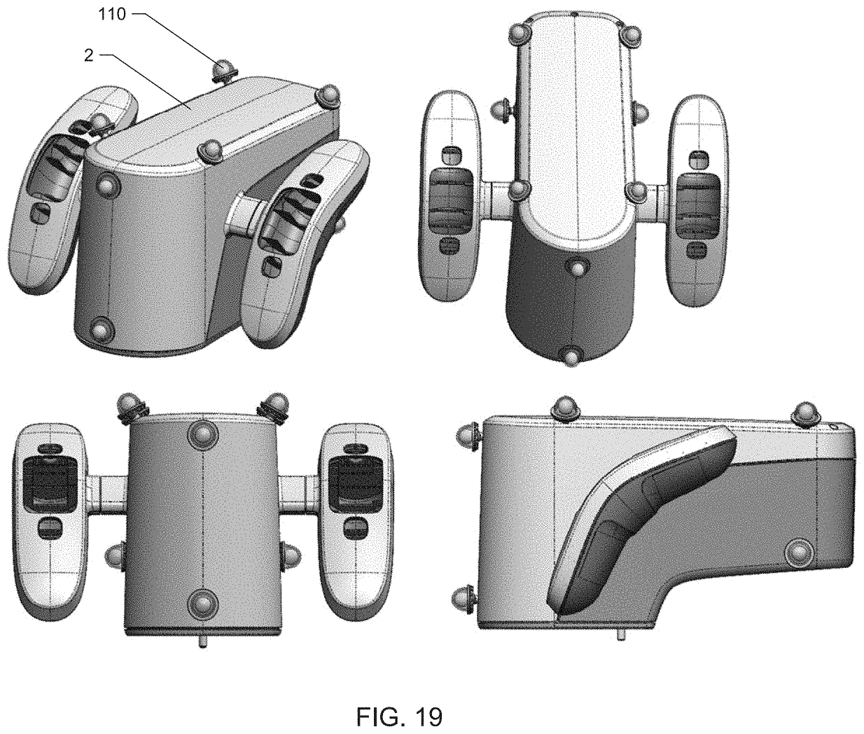

FIG. 19 is a diagram of another embodiment for camera tracking, according to an example embodiment of the present disclosure. In this embodiment, a housing of the camera 2 itself includes tracking features 110. As shown, the tracking features 110 are placed on multiple surfaces of the camera 2 to provide tracking from virtually any angle, orientation, or position. The top tracking features 110 are shown at intersections of the top surface and side surfaces, and are orientated at an angle between the surfaces. In other embodiments, the tracking features 110 may be placed in a middle of the surface. Further, while fiducials are shown, other embodiments can include cross points, as discussed above, or other tracking features.

### CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A shell apparatus for a surgical imaging camera, the apparatus comprising:
a top surface integrally formed with a front surface and two opposing side surfaces defining empty space therebetween, each of the top surface, the side surfaces, and the front surface including at least three tracking features;
at least six kinematic constraints located on an internally facing side of at least one of the top surface, the side surfaces, or the front surface; and
a connector that is positioned within an area defined by the at least six kinematic constraints.

2. The apparatus of claim 1, wherein the at least six kinematic constraints are located on an underside of the top surface.

3. The apparatus of claim 2, wherein the connector is located at the top surface.

4. The apparatus of claim 1, wherein each of the top surface, the side surfaces, and the front surface is configured to cradle or at least partially cover a housing of the surgical imaging camera within the empty space.

5. The apparatus of claim 4, wherein each of the at least six kinematic constraints is configured to mate or engage an object on a top surface of the surgical imaging camera.

6. The apparatus of claim 5, wherein the object includes at least one of a spherical object or semi-spherical object.

7. The apparatus of claim 4, wherein the connector is configured to engage a screw hole or receptor on the top surface of the surgical imaging camera.

8. The apparatus of claim 1, wherein the top surface, the side surfaces, and the front surface are configured to sandwich a surgical drape between the surgical imaging camera and the shell apparatus.

9. A shell apparatus for a surgical imaging camera, the apparatus comprising:
a top surface integrally formed with a front surface and two opposing side surfaces defining empty space therebetween, each of the top surface, the side surfaces, and the front surface including at least three tracking features; and
at least six kinematic constraints located on at least one of the top surface, the side surfaces, or the front surface.

10. The apparatus of claim 9, wherein the at least six kinematic constraints form points of a triangle.

11. The apparatus of claim 9, wherein each of the at least six kinematic constraints or grooves is configured to mate or engage a magnetic object on a respective surface of the surgical imaging camera to create a magnetic coupling between the surgical imaging camera and the shell apparatus.

12. The apparatus of claim 11, wherein the object includes at least one of a spherical object or semi-spherical object.

13. The apparatus of claim 9, further comprising at least one magnetic connector configured to magnetically couple to a corresponding at least one magnetic connector on the surgical imaging camera.

14. The apparatus of claim 9, wherein the two opposing side surfaces each have a boot shape.

15. The apparatus of claim 9, wherein at least one of the top surface, the side surfaces, or the front surface includes at least one window for weight reduction or heat dissipation.

16. The apparatus of claim 9, wherein each of the at least six kinematic constraints are orientated at different angles with respect to each other.

17. The apparatus of claim 9, wherein each of the top surface, the side surfaces, and the front surface includes protrusions configured to accept or support a respective tracking feature.

18. The apparatus of claim 17, wherein each of the protrusions extend orthogonally from the respective surface.

19. The apparatus of claim 17, wherein each of the protrusions and the respective tracking feature has a circular shape or an oval shape.

20. The apparatus of claim 1, wherein each of the tracking features includes at least one of a cross point, a fiducial, or a target.

21. The apparatus of claim 1, wherein each of the top surface, the side surfaces, and the front surface are constructed from plastic, carbon fiber, fiberglass, nylon, metal, or composites thereof.
